(19) United States
(12) Patent Application Publication
(10) Pub. No.: US 2012/0059034 A1
(43) Pub. Date: Mar. 8, 2012

(54) NOVEL CRYSTALLINE HYDRATE, AMORPHOUS AND POLYMORPHIC FORMS OF DIHYDRO-BENZOXAZOLE-6-YL-ACETAMIDE DERIVATIVE AND PROCESSES FOR THEIR PREPARATION

(76) Inventors: Ádám Demeter, Budapest (HU); Zoltán Német, Budapest (HU); András Nemes, Budapest (HU); Ferenc Sebok, Mezökovácsháza (HU); Gizella Bartáné Szalai, Budapest (HU); László Czibula, Budapest (HU)

(21) Appl. No.: 13/147,181

(22) PCT Filed: Mar. 1, 2010
(86) PCT No.: PCT/HU10/00023

§ 371 Date: Jul. 29, 2011

(30) Foreign Application Priority Data
Mar. 3, 2009 (HU) P09 00130

(51) Int. Cl. A61K 31/454; A61P 11/00; A61P 25/00; A61P 25/36; A61P 25/32; A61P 25/28; A61P 25/16; A61P 25/04; A61P 25/22; A61P 25/24; A61P 11/06; A61P 27/06; A61P 25/08; A61P 27/16; C07D 413/12

(52) U.S. Cl. 514/321; 546/198

(57) ABSTRACT

The invention relates to novel crystalline hydrate, amorphous and crystalline polymorphic forms of 2-[4-(4-Fluoro-benzyl)-piperidine-1-yl]-2-oxo-N-(2-oxo-2,3-dihydro-benzoxazole-6-yl)-acetamide (I) (radiprodil). Processes for the preparation of these forms, compositions containing these forms, and methods of use thereof are also described.

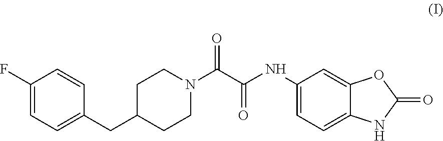

### FIELD OF THE INVENTION

The present invention relates to novel crystalline hydrates, amorphous and crystalline polymorphic forms of 2-[4-(4-Fluoro-benzyl)-piperidine-1-yl]-2-oxo-N-(2-oxo-2,3-dihydro-benzoxazole-6-yl)-acetamide (I) (radiprodil). Processes for the preparation of these forms, compositions containing these forms, and methods of use thereof are also described.

### BACKGROUND OF THE INVENTION

N-methyl-D-aspartate (NMDA) receptors are ligand-gated cation-channels embedded in the cell membranes of neurons. Overactivation of NMDA receptors by glutamate, their natural ligand, can lead to calcium overload of cells. This triggers a cascade of intracellular events that alters the cell function and ultimately may lead to death of neurons [TINS, 10, 299-302 (1987)]. Antagonists of the NMDA receptors may be used for treating many disorders that are accompanied with excess release of glutamate, the main excitatory neurotransmitter in the central nervous system. Moreover, NR2B subtype selective antagonists of NMDA receptors are expected to possess little or no untoward side effects that are typically caused by the non-selective antagonists of NMDA receptors, namely psychotomimetic effects such as dizziness, headache, hallucinations, dysphoria and disturbances of cognitive and motor function.

International Publication No. WO/2003/010159 discloses novel piperidine derivatives as NR2B subtype selective antagonists of NMDA receptors. One particular carboxylic acid amide derivative is the 2-[4-(4-Fluoro-benzyl)-piperidine-1-yl]-2-oxo-N-(2-oxo-2,3-dihydro-benzoxazole-6-yl)-acetamide (I), also known as radiprodil, is a highly effective NR2B subtype selective antagonists of NMDA receptors used for treatment of chronic neuropathic pain. The structural formula of radiprodil is shown below in figure (I).

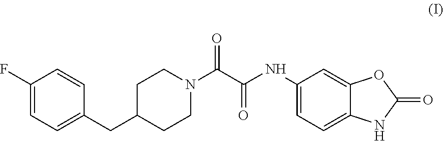

The present invention relates to the solid state physical properties of 2-[4-(4-Fluoro-benzyl)-piperidine-1-yl]-2-oxo-N-(2-oxo-2,3-dihydro-benzoxazole-6-yl)-acetamide (I). These properties may be influenced by controlling the conditions under which this compound is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. Distinct crystal structures have characteristic reflections with more or less characteristic relative intensities on their X-ray powder diffraction (XRPD) pattern, which usually permits unequivocal identification of polymorphic forms. The modification may give rise to thermal behavior different from that of the amorphous material or another modification. Thermal behavior is measured in the laboratory by such techniques as capillary melting point, thermogravimetric analysis (TGA) and differential scanning calorimetry (DSC) and may be used to distinguish some polymorphic forms from others. A particular solid phase may also give rise to distinct spectroscopic properties that may be detectable by solid state nuclear magnetic resonance (NMR) spectrometry, Raman spectroscopy and infrared (IR) spectrometry.

In deciding which polymorph is preferable from a certain pharmaceutical point of view, the numerous physical, technological and biological properties of the polymorphs must be compared. It is entirely possible that one polymorph can be preferable in some circumstances in which certain aspects, such as ease of preparation, filtration, chemical purity, stability, etc., are deemed to be critical. In other situations, a different polymorph may be preferred for greater solubility and/or superior pharmacokinetics. Again, in other cases, a particular polymorph may be preferred from the formulation point of view leading to drug product.

The synthesis of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. New polymorphic forms, hydrates and amorphous form of 2-[4-(4-Fluoro-benzyl)-piperidine-1-yl]-2-oxo-N-(2-oxo-2,3-dihydro-benzoxazole-6-yl)-acetamide have now been synthetized.

Processes for preparing 2-[4-(4-Fluoro-benzyl)-piperidine-1-yl]-2-oxo-N-(2-oxo-2,3-dihydro-benzoxazole-6-yl)-acetamide (I), also known as radiprodil are described in WO/2003/010159 in example 3. According to Example 3, column chromatography with chloroform:methanol=19:1 as eluent is used in the final purification step, after which the purified residue is triturated with diethylether. This procedure affords a solid form having a stated melting point of 224-227° C.

### SUMMARY OF THE INVENTION

The present invention relates to novel crystalline hydrate, amorphous and crystalline polymorphic forms of 2-[4-(4-Fluoro-benzyl)-piperidine-1-yl]-2-oxo-N-(2-oxo-2,3-dihydro-benzoxazole-6-yl)-acetamide (I). Processes for the preparation of these forms, compositions containing these forms, and methods of use thereof are also described.

### BRIEF DESCRIPTION OF THE FIGURES

FIG. 1 shows the X-ray powder diffraction pattern of anhydrate Form A of 2-[4-(4-Fluoro-benzyl)-piperidine-1-yl]-2-oxo-N-(2-oxo-2,3-dihydro-benzoxazole-6-yl)-acetamide (radiprodil anhydrate Form A).

FIG. 2 shows the FT-IR spectrum of anhydrate Form A 2-[4-(4-Fluoro-benzyl)-piperidine-1-yl]-2-oxo-N-(2-oxo-2,3-dihydro-benzoxazole-6-yl)-acetamide (radiprodil anhydrate Form A).

FIG. 3 shows the FT-Raman spectrum of anhydrate Form A of 2-[4-(4-Fluoro-benzyl)-piperidine-1-yl]-2-oxo-N-(2-oxo-2,3-dihydro-benzoxazole-6-yl)-acetamide (radiprodil anhydrate Form A).

FIG. 4 shows the thermogravimetric analysis curve of anhydrate Form A of 2-[4-(4-Fluoro-benzyl)-piperidine-1-yl]-2-oxo-N-(2-oxo-2,3-dihydro-benzoxazole-6-yl)-acetamide (radiprodil anhydrate Form A).

FIG. 5 shows the differential scanning calorimetry trace of anhydrate Form A of 2-[4-(4-Fluoro-benzyl)-piperidine-1-yl]-2-oxo-N-(2-oxo-2,3-dihydro-benzoxazole-6-yl)-acetamide (radiprodil anhydrate Form A).

FIG. 6 shows the X-ray powder diffraction pattern of anhydrate Form B of 2-[4-(4-Fluoro-benzyl)-piperidine-1-yl]-2-oxo-N-(2-oxo-2,3-dihydro-benzoxazole-6-yl)-acetamide (radiprodil anhydrate Form B).

FIG. 7 shows the FT-IR spectrum of anhydrate Form B of 2-[4-(4-Fluoro-benzyl)-piperidine-1-yl]-2-oxo-N-(2-oxo-2,3-dihydro-benzoxazole-6-yl)-acetamide (radiprodil anhydrate Form B).

FIG. 8 shows the FT-Raman spectrum of anhydrate Form B of 2-[4-(4-Fluoro-benzyl)-piperidine-1-yl]-2-oxo-N-(2-oxo-2,3-dihydro-benzoxazole-6-yl)-acetamide (radiprodil anhydrate Form B).

FIG. 9 shows the thermogravimetric analysis curve of anhydrate Form B of 2-[4-(4-Fluoro-benzyl)-piperidine-1-yl]-2-oxo-N-(2-oxo-2,3-dihydro-benzoxazole-6-yl)-acetamide (radiprodil anhydrate Form B).

FIG. 10 shows the differential scanning calorimetry trace of anhydrate Form B of 2-[4-(4-Fluoro-benzyl)-piperidine-1-yl]-2-oxo-N-(2-oxo-2,3-dihydro-benzoxazole-6-yl)-acetamide (radiprodil anhydrate Form B).

FIG. 11 shows the X-ray powder diffraction pattern of 2-[4-(4-Fluoro-benzyl)-piperidine-1-yl]-2-oxo-N-(2-oxo-2,3-dihydro-benzoxazole-6-yl)-acetamide dihydrate (radiprodil dihydrate).

FIG. 12 shows the FT-IR spectrum of 2-[4-(4-Fluoro-benzyl)-piperidine-1-yl]-2-oxo-N-(2-oxo-2,3-dihydro-benzoxazole-6-yl)-acetamide dihydrate (radiprodil dihydrate).

FIG. 13 shows the FT-Raman spectrum of 2-[4-(4-Fluoro-benzyl)-piperidine-1-yl]-2-oxo-N-(2-oxo-2,3-dihydro-benzoxazole-6-yl)-acetamide dihydrate (radiprodil dihydrate).

FIG. 14 shows the thermogravimetric analysis curve of 2-[4-(4-Fluoro-benzyl)-piperidine-1-yl]-2-oxo-N-(2-oxo-2,3-dihydro-benzoxazole-6-yl)-acetamide dihydrate (radiprodil dihydrate).

FIG. 15 shows the differential scanning calorimetry trace of 2-[4-(4-Fluoro-benzyl)-piperidine-1-yl]-2-oxo-N-(2-oxo-2,3-dihydro-benzoxazole-6-yl)-acetamide dihydrate (radiprodil dihydrate).

FIG. 16 shows the X-ray powder diffraction pattern of 2-[4-(4-Fluoro-benzyl)-piperidine-1-yl]-2-oxo-N-(2-oxo-2,3-dihydro-benzoxazole-6-yl)-acetamide monohydrate (radiprodil monohydrate).

FIG. 17 shows the FT-IR spectrum of 2-[4-(4-Fluoro-benzyl)-piperidine-1-yl]-2-oxo-N-(2-oxo-2,3-dihydro-benzoxazole-6-yl)-acetamide monohydrate (radiprodil monohydrate).

FIG. 18 shows the FT-Raman spectrum of 2-[4-(4-Fluoro-benzyl)-piperidine-1-yl]-2-oxo-N-(2-oxo-2,3-dihydro-benzoxazole-6-yl)-acetamide monohydrate (radiprodil monohydrate).

FIG. 19 shows the thermogravimetric analysis curve of 2-[4-(4-Fluoro-benzyl)-piperidine-1-yl]-2-oxo-N-(2-oxo-2,3-dihydro-benzoxazole-6-yl)-acetamide monohydrate (radiprodil monohydrate).

FIG. 20 shows the differential scanning calorimetry trace of 2-[4-(4-Fluoro-benzyl)-piperidine-1-yl]-2-oxo-N-(2-oxo-2,3-dihydro-benzoxazole-6-yl)-acetamide monohydrate (radiprodil monohydrate).

FIG. 21 shows the X-ray powder diffraction pattern of the amorphous 2-[4-(4-Fluoro-benzyl)-piperidine-1-yl]-2-oxo-N-(2-oxo-2,3-dihydro-benzoxazole-6-yl)-acetamide (radiprodil amorphous).

FIG. 22 shows the FT-IR spectrum of the amorphous 2-[4-(4-Fluoro-benzyl)-piperidine-1-yl]-2-oxo-N-(2-oxo-2,3-dihydro-benzoxazole-6-yl)-acetamide (radiprodil amorphous).

FIG. 23 shows the FT-Raman spectrum of the amorphous 2-[4-(4-Fluoro-benzyl)-piperidine-1-yl]-2-oxo-N-(2-oxo-2,3-dihydro-benzoxazole-6-yl)-acetamide (radiprodil amorphous).

FIG. 24 shows the thermogravimetric analysis curve of the amorphous 2-[4-(4-Fluoro-benzyl)-piperidine-1-yl]-2-oxo-N-(2-oxo-2,3-dihydro-benzoxazole-6-yl)-acetamide (radiprodil amorphous).

FIG. 25 shows the differential scanning calorimetry trace of the amorphous 2-[4-(4-Fluoro-benzyl)-piperidine-1-yl]-2-oxo-N-(2-oxo-2,3-dihydro-benzoxazole-6-yl)-acetamide (radiprodil amorphous).

### DETAILED DESCRIPTION OF THE INVENTION

The present invention relates to novel crystalline hydrate, amorphous and polymorphic forms of 2-[4-(4-Fluoro-benzyl)-piperidine-1-yl]-2-oxo-N-(2-oxo-2,3-dihydro-benzoxazole-6-yl)-acetamide (I) (radiprodil) and methods for the preparation of it. Polymorphism is the ability of any element or compound to crystallize as more than one distinct crystal species. Different polymorphs of a given compound are, in general, as different in structure and properties, as the crystals of two different compounds. Solubility, melting point, density, hardness, crystal shape, optical and electrical properties, vapor pressure, etc., all vary with the polymorphic form. Pharmaceutical solids as active pharmaceutical ingredients and excipients are generally molecular crystals which are very prone to form polymorph modifications. Of the several contributory factors to the bioavailability of finished products, the inherent solubility and rate of dissolution of the drug substance itself are of major importance. The inherent solubility and the rate of dissolution of an active ingredient in a patient's stomach fluid may have therapeutic consequences since it imposes an upper limit on the rate at which an orally-administered active ingredient may reach the patient's bloodstream. By choosing polymorphs with different solubility and/or intrinsic dissolution rate, the actual blood level of the drug can be favorably affected. The rate of dissolution is also a consideration from a formulation point of view. The solubility is dependent on the polymorphic state, as different polymorphs have different free energies and therefore different solubilities. Those who are skilled in the art know that amorphous compounds, due to their higher energy compared to crystalline forms, generally show the largest solubility and intrinsic dissolution rate, therefore active ingredients in an amorphous form may be advantageous for the formulation of fast release product forms with better bioavailability. One particular drawback of amorphous forms is their lower physical stability compared to crystalline forms, i.e. amorphous forms are prone to crystallize in time.

Preparing stable amorphous form of an active ingredient can be as important as finding new polymorphic forms. Those who are skilled in the art would know that stability and other solid state properties of amorphous forms are dependent on the process of preparation.

Different physicochemical properties of polymorphic forms may also affect processing of the drug substance and/or manufacturing of the drug product. For a drug product manufactured by direct compression, the solid state properties of the active ingredient will likely be critical to the manufacture of the drug product, particularly when it constitutes the bulk of the tablet mass. These properties may be influenced by controlling the conditions under which this compound is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Polymorphic forms of the drug substance can undergo phase conversion when exposed to a range of manufacturing processes, such as drying, milling, micronization, wet granulation, spray-drying, and compaction. Exposure to environmental conditions such as humidity and temperature can also induce polymorph conversion. The extent of conversion generally depends on the relative stability of the polymorphs, kinetic barriers to phase conversion, and applied stress. Nonetheless, phase conversion generally is not of serious concern, provided that the conversion occurs consistently, as a part of a validated manufacturing process where critical manufacturing process variables are well understood and controlled, and when drug product bioavailability and bioequivalence has been demonstrated.

In deciding which polymorph is preferable from a certain pharmaceutical point of view, the numerous physical, technological and biological properties of the polymorphs must be compared. It is entirely possible that one polymorph can be preferable in some circumstances in which certain aspects, such as ease of preparation, filtration, chemical purity, stability, etc., are deemed to be critical. In other situations, a different polymorph may be preferred for greater solubility and/or superior pharmacokinetics. Again, in other cases, a particular polymorph may be preferred from the formulation point of view leading to drug product.

In view of the pharmaceutical value of a compound, it is of prime importance to obtain it with excellent purity. It is also important to be able to synthesize it by means of a process that can readily be converted to industrial scale, especially in a form that allows rapid filtration and drying. From the above technological and product quality point of view certain polymorphs, via a specific synthetic route, provide unique opportunity to fulfill these requirements. Therefore there is a pharmaceutical need to find proper polymorphs and crystallization process that is advantageously provide a compound in excellent purity with rapid filtration and drying properties in an industrial scale.

In other situations, a different polymorph may be preferred for greater solubility and/or superior pharmacokinetics. The synthesis of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

The different conformation and orientation of molecules in the unit cell, which defines a particular polymorphic form of a substance is resulted in different physical properties, which permit the solid state analytical characterization of these phases. Distinct crystal structures have characteristic reflections with more or less characteristic relative intensities on their X-ray powder diffraction pattern, which usually permits unequivocal identification of polymorphic forms. The modification may give rise to thermal behavior different from that of the amorphous material or another modification. Thermal behavior is measured in the laboratory by such techniques as capillary melting point, thermogravimetric analysis (TGA) and differential scanning calorimetry (DSC) and may be used to distinguish some polymorphic forms from others. A particular solid phase may also give rise to distinct spectroscopic properties that may be detectable by solid state nuclear magnetic resonance (NMR) spectrometry, Raman spectroscopy and infrared (IR) spectrometry. These analytical techniques therefore are suitable to characterize polymorphic forms.

According to the facts described above there is a need to find new polymorphic forms of a pharmaceutical compound which might be preferable for a number of different properties.

Processes for preparing 2-[4-(4-Fluoro-benzyl)-piperidine-1-yl]-2-oxo-N-(2-oxo-2,3-dihydro-benzoxazole-6-yl)-acetamide (I) radiprodil are described in WO/2003/010159 in example 3. According to Example 3, column chromatography with chloroform:methanol=19:1 as eluent is used in the final purification step, after which the purified residue is triturated with diethylether. This procedure affords a solid form having a stated melting point of 224-227° C. The chemical purity of the product should be improved to obtain the active ingredient in sufficiently high quality required for development of a product for human use. Therefore there is a need to find new polymorphic forms in chemically and physically pure form and an industrial processes leading to new solid forms that can be advantageous for different pharmaceutical reasons as explained above. To solve this need we have carried out a thorough research to find new polymorphic forms of radiprodil. Those of ordinary in the art would know that an a priori knowledge of what kind of polymorphs of a given compound might exist and what process leads to their formation does not exist. This should be discovered by a purely empirical process which is the subject of the present invention.

We have surprisingly found that radiprodil is prone to exhibit polymorphism. We have discovered, synthesized and thoroughly characterized new polymorphs and amorphous forms of radiprodil which might be advantageous in different ways as detailed below. We have surprisingly found also that radiprodil can form hydrates with varying stoichiometry, i.e. radiprodil *nH₂O, where n is equal to 0.5, 1, 1.5, 2.

### Radiprodil Anhydrate Form A

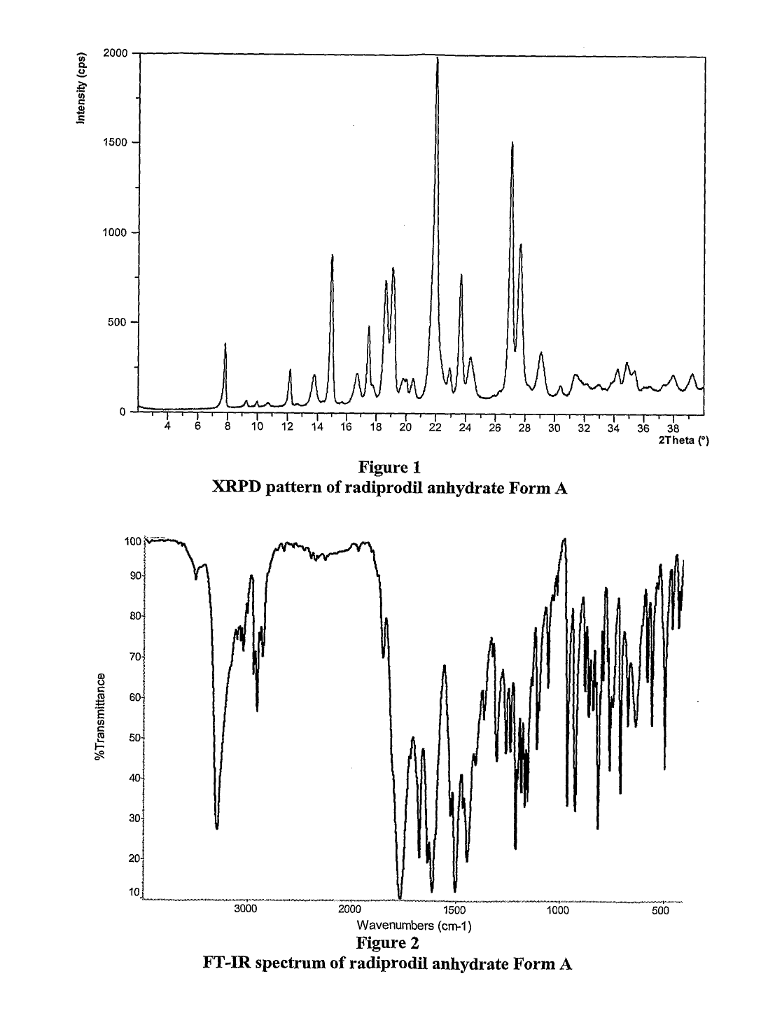

In accordance with the present invention it has been found that the anhydrate Form A of radiprodil is produced from an organic solvent or from a water miscible organic solvent by water feeding. Solid state characterization (XRPD, IR, Raman, DSC, TO) of the anhydrate Form A of radiprodil is given in FIG. 1-5, respectively. Anhydrate Form A is characterized by one or more of the following solid state features:

an X-ray powder diffraction pattern substantially in accordance with FIG. 1;

(I) an FT-IR spectrum substantially in accordance with FIG. 2;

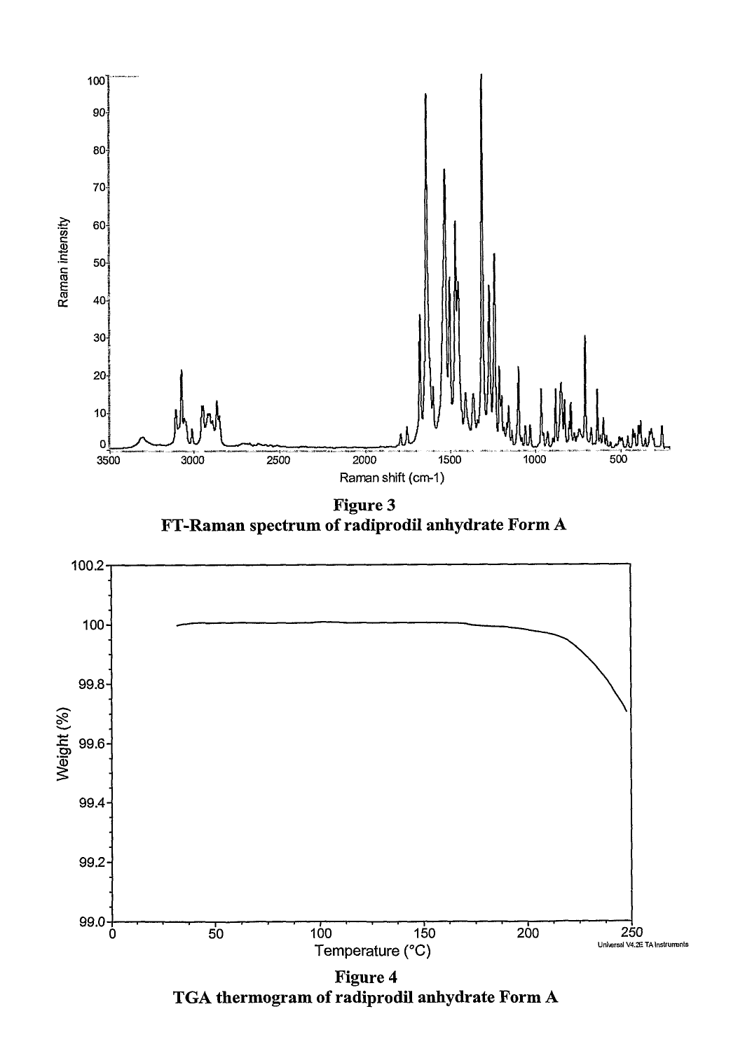

(II) an FT-Raman spectrum substantially in accordance with FIG. 3;

(III) a TGA thermogram substantially in accordance with FIG. 4;

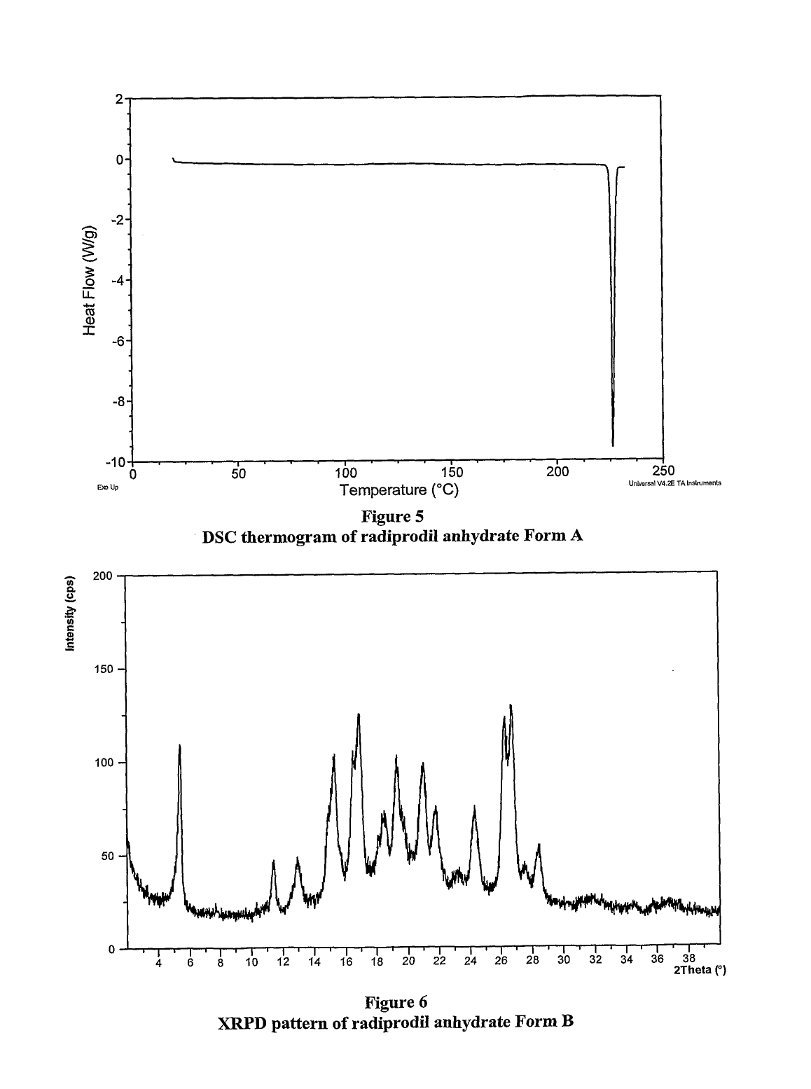

(IV) a DSC trace substantially in accordance with FIG. 5;
Characteristic diffraction and spectroscopic data of radiprodil anhydrate Form A are as follows:
   1. Characteristic XRPD reflections of radiprodil anhydrate Form A are the following: 7.8, 22.0, 23.7, 27.0 and 27.6±0.2° 2 θ
   2. Characteristic IR absorption bands of radiprodil anhydrate Form A are the following: 3295, 2863, 1852, 1768, 1058 and 635±4 cm⁻¹.
   3. Characteristic Raman absorption bands of radiprodil anhydrate Form A are the following: 3106, 1680, 1641, 1470, 1274 and 507±4 cm⁻¹.
   The anhydrate Form A of radiprodil has the advantage that at room temperature it is the thermodynamically most stable form discovered so far. All the other discovered solid forms can be converted to anhydrate Form A in suspension. This conversion takes place in absolute ethanol or in ethanol:water mixtures within a few hours. This relative stability advantage of anhydrous Form A of radiprodil can be favorably utilized in the development of active ingredient as well as dosage forms that are sufficiently stable both chemically and physically during extended shelf life. By exposing anhydrate Form A to stress stability conditions (25° C./75% RH; 50° C.; 50° C./oxygen; 50° C./75% RH; 50° C./90% RH; 75° C.; 75° C./oxygen) for 10 days neither the sum of chemical impurities nor the physical form has changed as compared to the starting material. Another technological advantage of anhydrate Form A is that this form is non-hygroscopic, it did not show measurable weight gain after exposing it to elevated humidity conditions (50° C./90% RH, 10 days). Due to its physical stability and non-hygroscopic character this polymorphic form is particularly advantageous in dosage formulation processes where the active ingredient is exposed to water during the process, such as wet granulation.

### Radiprodil Anhydrate Form B

In accordance with the present invention it has been found that the anhydrous Form B of radiprodil is produced from the monohydrate by heating at elevated temperature, preferably up to 130° C. The anhydrate Form B of radiprodil is characterized by its TG, DSC thermograms, by its IR and Raman spectra as well as X-ray powder diffraction pattern (FIG. 6-10). Anhydrate Form B is characterized by one or more of the following solid state features:

(I) an X-ray powder diffraction pattern substantially in accordance with FIG. 6;

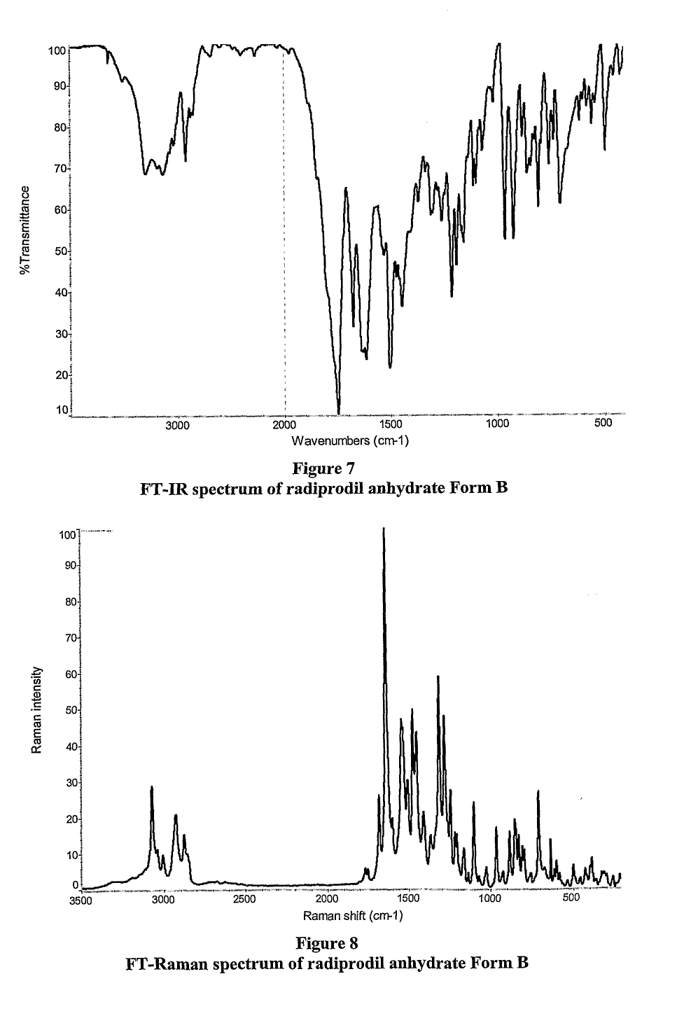

(II) an FT-IR spectrum substantially in accordance with FIG. 7;

(III) an FT-Raman spectrum substantially in accordance with FIG. 8;

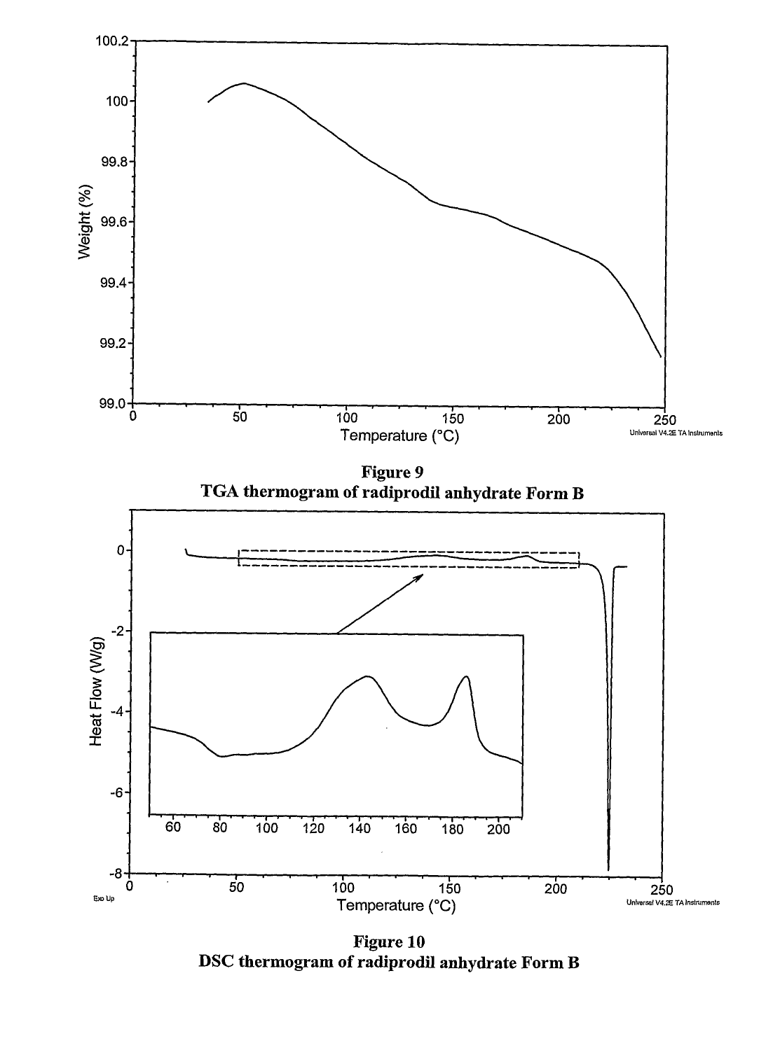

(IV) a TGA thermogram substantially in accordance with FIG. 9

(V) a DSC trace substantially in accordance with FIG. 10;

Characteristic diffraction and spectroscopic data of radiprodil anhydrate Form B are as follows:

1. Characteristic XRPD reflections of radiprodil anhydrate Form B are the followings: 5.4, 12.9, 16.9 and 21.0±0.2° 2θ.
2. Characteristic IR absorption bands of radiprodil anhydrate Form B are the followings: 3138, 1752, 809 and 559±4 cm⁻¹.
3. Characteristic Raman absorption bands of radiprodil anhydrate Form B are the followings: 2877, 1684, 1642, 1545, 1476 and 887±4 cm⁻¹.

### Radiprodil Dihydrate

In accordance with the present invention it has been found that the dihydrate form of radiprodil is produced by adding solution of radiprodil in an organic solvent to dilute aqueous NaHCO₃ solution. It is worth noting that inverted dosage or crystallization from anhydric or hydric organic solvent led to radiprodil anhydrate Form A.

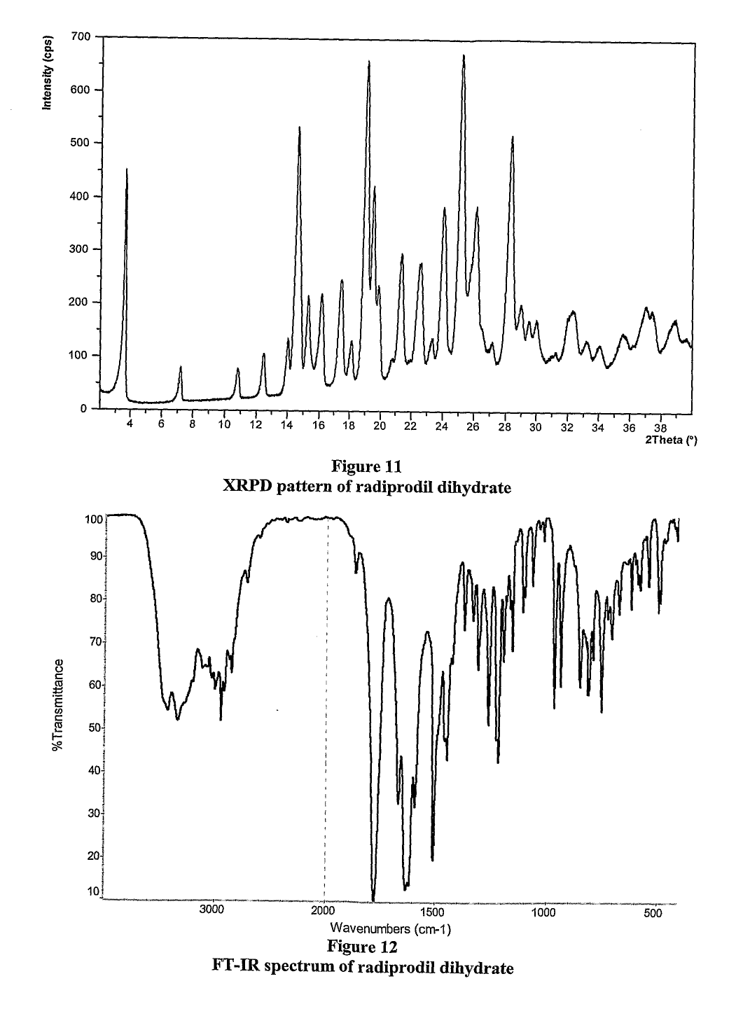

The dihydrate Form of radiprodil is characterized by its Karl-Fischer water content of about 7-9%, TG, DSC thermograms, by its IR and Raman spectra as well as X-ray powder diffraction pattern (FIG. 11-15).
The dihydrate Form is characterized by one or more of the following solid state features:

(I) an X-ray powder diffraction pattern substantially in accordance with FIG. 11;

(II) an FT-IR spectrum substantially in accordance with FIG. 12;

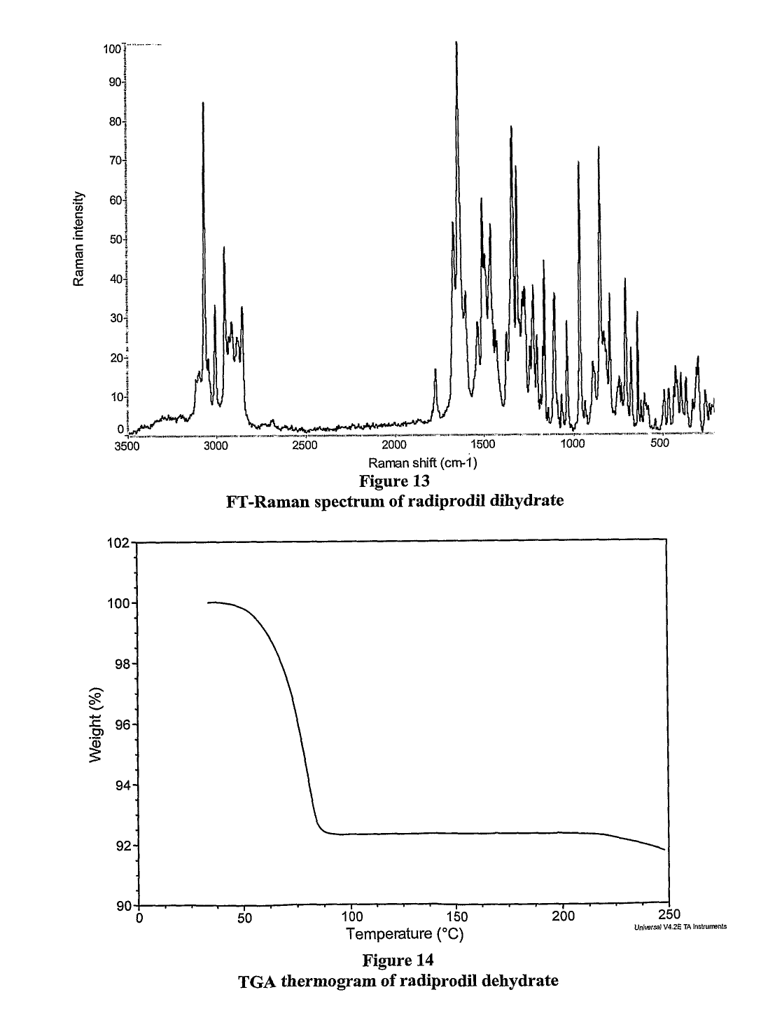

(III) an FT-Raman spectrum substantially in accordance with FIG. 13;

(IV) a TGA thermogram substantially in accordance with FIG. 14;

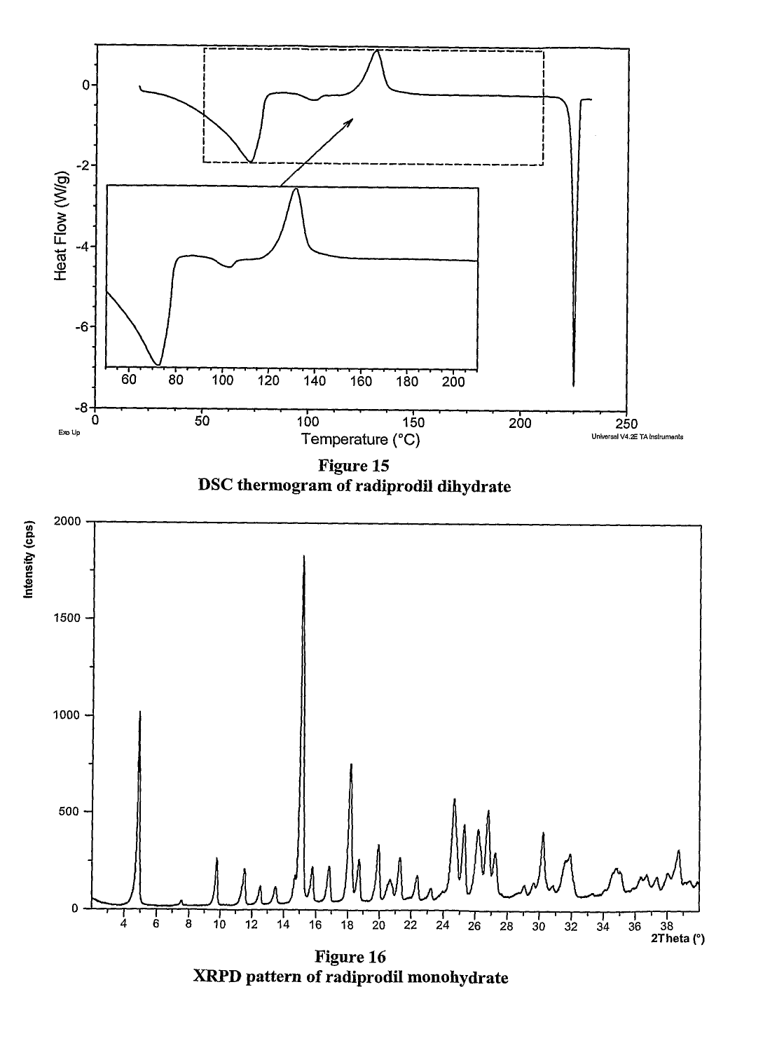

(V) a DSC trace substantially in accordance with FIG. 15;

Characteristic diffraction and spectroscopic data of radiprodil dihydrate are as follows:
1. Characteristic XRPD reflections of radiprodil dihydrate are the followings: 3.6, 14.7, 19.1, 25.2 and 28.3±0.2° 2θ.
2. Characteristic IR absorption bands of radiprodil dihydrate are the followings: 3426, 2950, 1870, 1776, 1668 and 754±4 cm⁻¹.
3. Characteristic Raman absorption bands of radiprodil dihydrate are the followings: 3068, 1772, 1669, 1641 and 1313±4 cm⁻¹.

It has now been discovered, that dihydrate Form of radiprodil is particularly stable and hence is suitable for bulk preparation and handling. This form shows a cracked plate-like habit, which allows rapid filtration and drying, finally it can be prepared by an efficient, economic and reproducible process, providing a product of excellent purity. One particular advantage of the crystalline dihydrate Form is that it provides radiprodil in the highest chemical purity which makes this solid form superior for using as an active ingredient in dosage forms for human use. Another essential value of the dihydrate Form is the observed better pharmacokinetic profile in dogs compared to the anhydrate Form A. Both forms in 5 mg/kg dose adjusted to the anhydrous active substance were administered to dogs and relevant pharmacokinetic parameters (AUC, Cₘₐₓ) were calculated from the plasma profiles of the individual animals. The animals were fed seventeen hours before and six hours after drug administration. Plasma concentrations were measured in the trial samples taken up to 24 hours post dose by a validated HPLC(UV) method. It was found that dihydrate Form shows better bioavailability (AUC₂₄=4491 ng/ml*h, Cₘₐₓ=395 ng/ml) as compared to the anhydrate Form A (AUC₂₄=1554 ng/ml*h, Cₘₐₓ=125 ng/ml). These results underpin our discovery that the dihydrate Form is particularly advantageous as an active ingredient in dosage forms.

### Radiprodil Monohydrate

In accordance with the present invention it has been found that the monohydrate form of radiprodil is produced from acetone, containing 5-10% of water. The monohydrate is prepared by cooling a solution of radiprodil in an acetone/water mixture. On account of controlled temperature program the crystallization is initiated by concentrating the solution under reduced pressure at 35-40° C., followed by cooling to 20-25° C. and the crystal growth is completed by agitation at 0-5° C.

The monohydrate is characterized by a Karl-Fischer water content of about 3-5 wt. %, by its TG, DSC thermogram and by its IR and Raman spectra as well as X-ray powder diffraction pattern (FIG. 16-20).

The monohydrate Form is characterized by one or more of the following solid state features:

(I) an X-ray powder diffraction pattern substantially in accordance with FIG. 16;

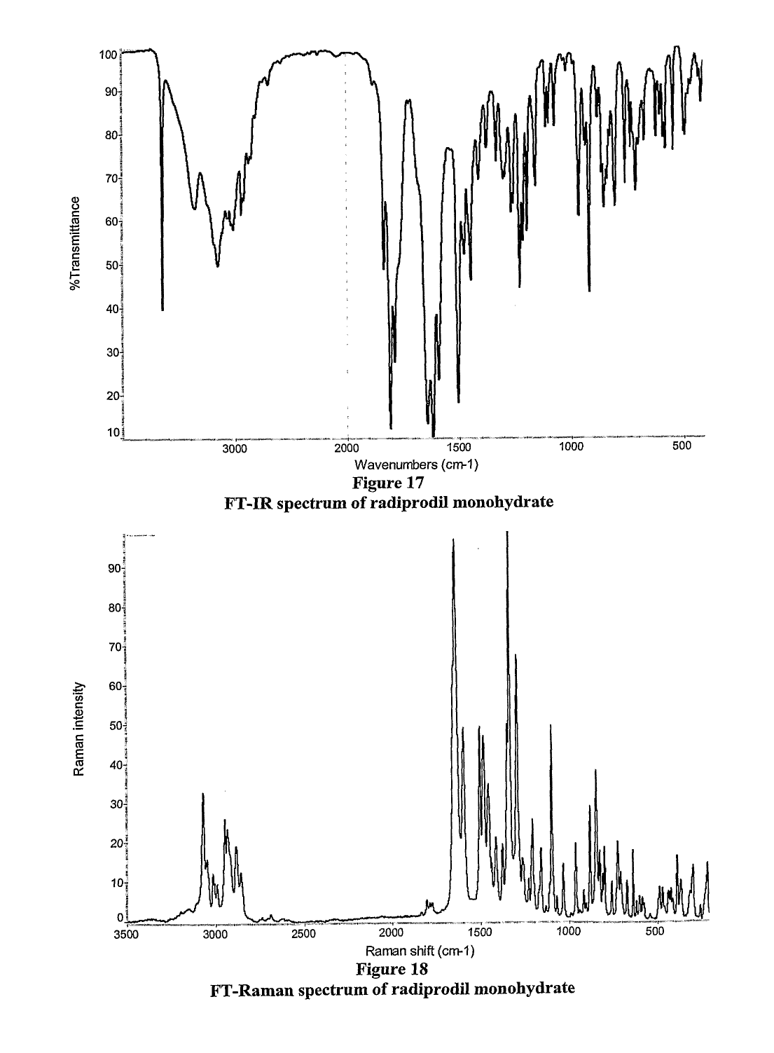

(II) an FT-IR spectrum substantially in accordance with FIG. 17;

(III) an FT-Raman spectrum substantially in accordance with FIG. 18;

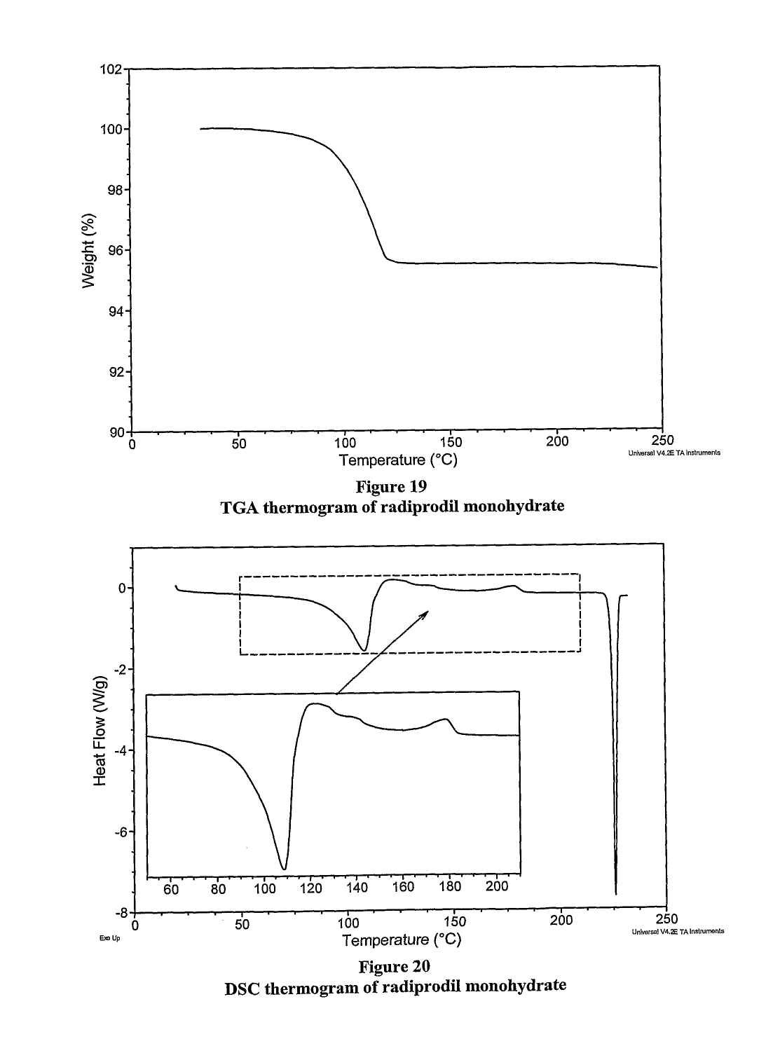

(IV) a TGA thermogram substantially in accordance with FIG. 19;

(V) a DSC trace substantially in accordance with FIG. 20;

Characteristic diffraction and spectroscopic data of radiprodil monohydrate are as follows:

1. Characteristic reflections of radiprodil monohydrate are the followings: 4.9, 15.1, 18.1, 26.8 and 30.2±0.2° 2θ
2. Characteristic IR absorption bands of radiprodil monohydrate are the followings: 3644, 1836, 1809, 1789, 1645 and 919±4 cm⁻¹.
3. Characteristic Raman absorption bands of radiprodil monohydrate are the followings: 2887, 1642, 1484, 1295 and 726±4 cm⁻¹.
The monohydrate Form exhibits larger thermal stability compared to the dihydrate Form. While the dihydrate Form can be dehydrated relatively easily at elevated temperature, the monohydrate Form is sufficiently stable under such conditions. At 50° C. in N₂ atmosphere the dihydrate Form loses its water of hydration in 2 hours, the monohydrate Form, however, does not show detectable weight loss under the same conditions, the latter keeps its water of hydration stable. This relative stability of the monohydrate Form compared to the dihydrate Form is advantageous in formulation technological steps involving elevated temperatures. The advantage of monohydrate Form over anhydrate Form A is its greater solubility due to lower thermodynamic stability, which may be favorable from pharmacokinetic aspects like in the case of dihydrate.

### Radiprodil Amorphous

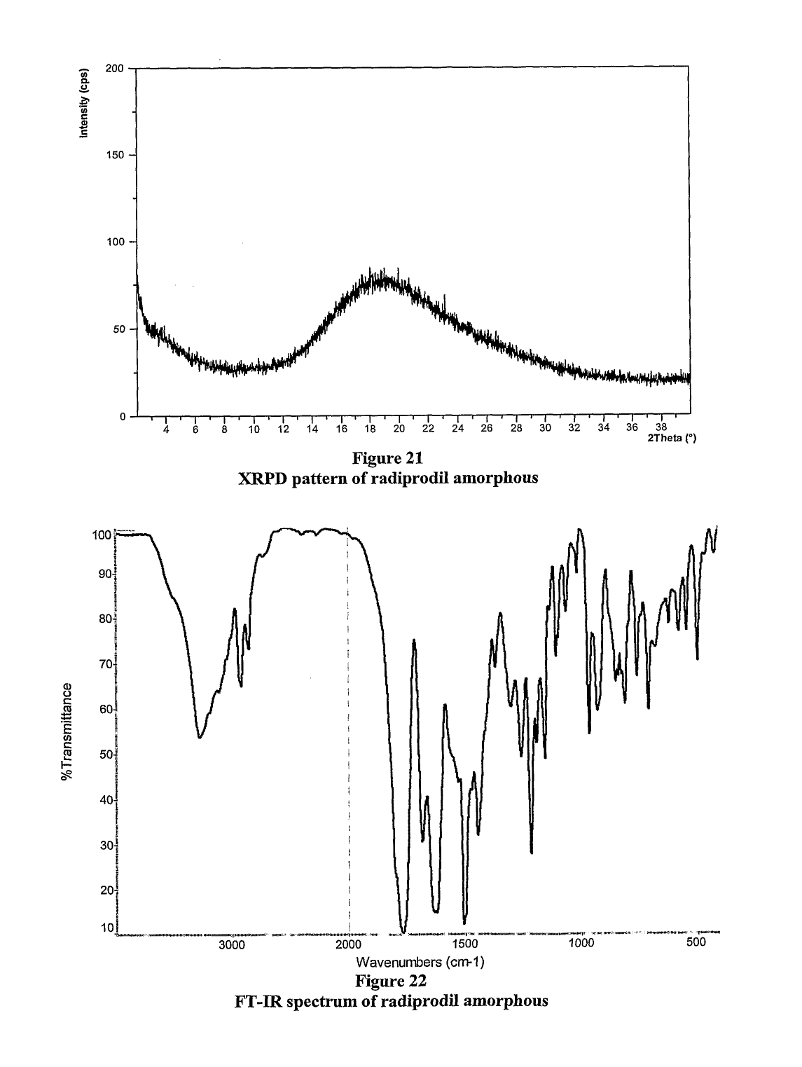

In accordance with the present invention it has been found that the amorphous form of radiprodil is produced by drying the dihydrate Form at about 100° C. under vacuo or dry nitrogen. The amorphous form is characterized by its TG, DSC thermogram, by its IR and Raman spectra as well as absence of reflections on X-ray powder diffraction pattern (FIG. 21-25).

The amorphous Form is characterized by one or more of the following solid state features:

(I) an X-ray powder diffraction pattern substantially in accordance with FIG. 21;

(II) an FT-IR spectrum substantially in accordance with FIG. 22;

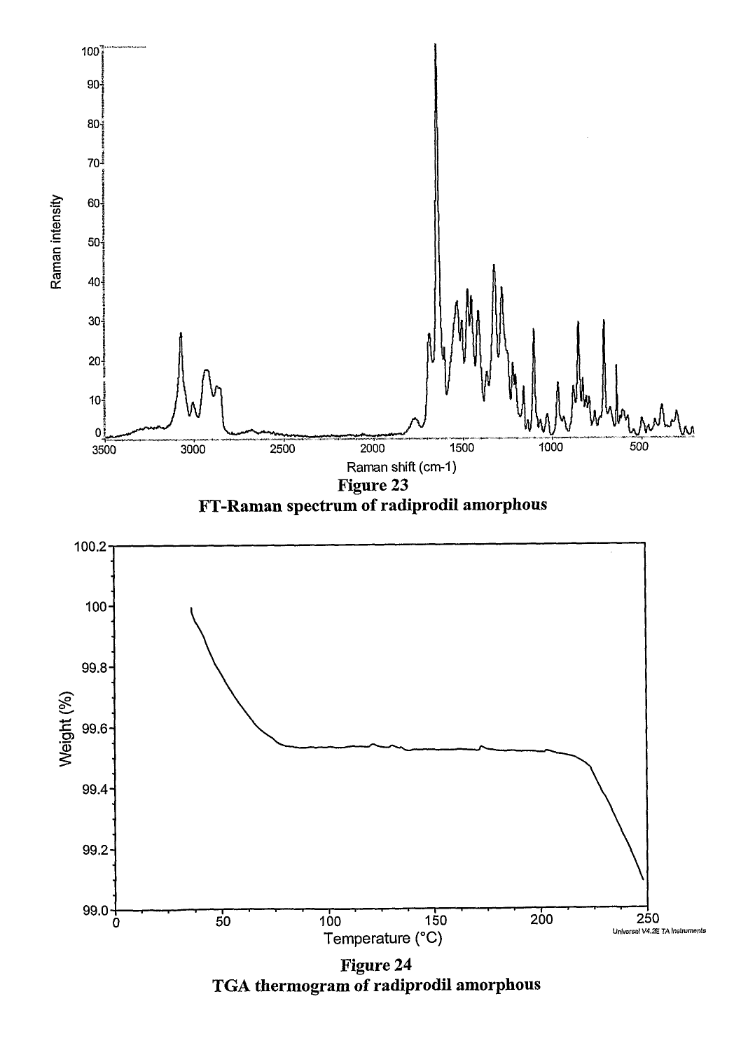

(III) an FT-Raman spectrum substantially in accordance with FIG. 23;

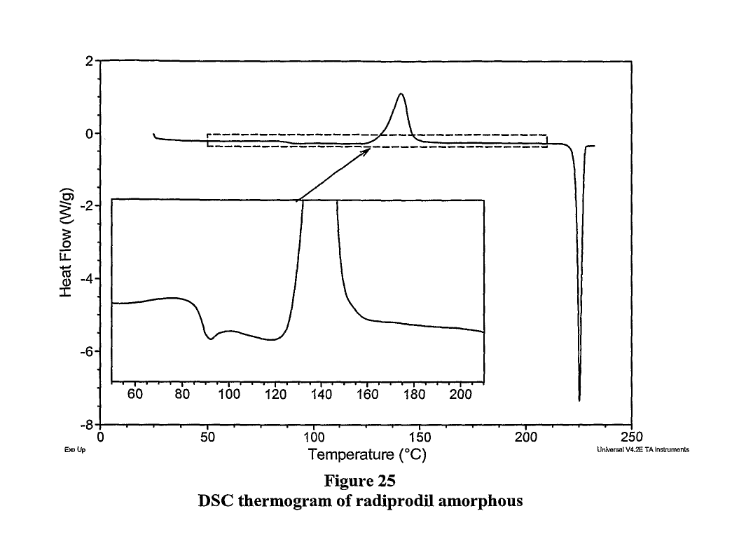

(IV) a DSC trace substantially in accordance with FIG. 25;

Characteristic diffraction and spectroscopic data of radiprodil amorphous are as follows:

1. There are no reflections on XRPD pattern of radiprodil amorphous form.
2. Characteristic IR absorption bands of radiprodil amorphous form are the followings: 3275, 1768, 1685, 1219 and 544±4 cm⁻¹.
3. Characteristic Raman absorption bands of radiprodil amorphous are the followings: 3071, 1687, 1640, 1414, 1280 and 852±4 cm⁻¹.
We have found that the amorphous Form shows the largest solubility and dissolution rate which is favorable to produce fast release dosage forms. One particular advantage of the discovered amorphous Form is that it is relatively stable. It can be stored without crystallization under normal laboratory conditions for several months, which provides an opportunity for developing stable amorphous dosage forms.

### Compositions

Solid forms of Radiprodil (anhydrate Form A, anhydrate Form B, dihydrate, monohydrate, amorphous) described above can be administered alone or as an active ingredient of a formulation. Thus, the present invention also includes pharmaceutical compositions of polymorphs and solvates of the invention, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of various solid forms of Radiprodil (anhydrate Form A, anhydrate Form B, dihydrate, monohydrate, amorphous) may be accomplished according to patient needs, for example, orally, nasally, parenterally (subcutaneously, intraveneously, intramuscularly, intrasternally and by infusion) by inhalation, rectally, vaginally, topically and by ocular administration.

Various solid oral dosage forms can be used for administering the polymorphs and solvates of the invention including such solid forms as tablets, gelcaps, capsules, caplets, granules, lozenges and bulk powders. The polymorphs and solvates of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering the polymorphs and solvates of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The polymorphs and solvates of the present invention may be injected, for example, intravenously, in the form of an isotonic sterile solution. Other preparations are also possible.

Suppositories for rectal administration of the polymorphs and solvates of the present invention can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

In one embodiment, the invention provides a composition comprising any of the above described solid forms of 2-[4-(4-fluoro-benzyl)-piperidine-1-yl]-2-oxo-N-(2-oxo-2,3-dihydro-benzoxazole-6-yl)-acetamide (Radiprodil anhydrate Form A, anhydrate Form B, dihydrate, monohydrate, and amorphous) and a pharmaceutically acceptable carrier.

The invention also provides the use of a compound of the present invention in the manufacture of a medicament for the treatment of conditions which require modulation of an NMDA receptor, e.g., an NR2B selective NMDA receptor.

The present invention further provides methods for treating conditions which require modulation of an NMDA receptor, e.g., an NR2B selective NMDA receptor, comprising administering an effective amount of any of the above described solid forms of 2-[4-(4-fluoro-benzyl)-piperidine-1-yl]-2-oxo-N-(2-oxo-2,3-dihydro-benzoxazole-6-yl)-acetamide (Radiprodil anhydrate Form A, anhydrate Form B, dihydrate, monohydrate, and amorphous).

In a further embodiment, the present invention further provides for the use of any of the above described solid forms of Radiprodil (anhydrate Form A, anhydrate Form B, dihydrate, monohydrate, and amorphous) in the manufacture of a medicament for the treatment and/or prevention of conditions which requires modulation of an NMDA receptor, e.g., an NR2B selective NMDA receptor.

Disorders which may be beneficially treated with NMDA antagonists include, for example, traumatic injury of brain [Neurol. Res., 21, 330-338 (1999)] or spinal cord [Eur. J. Pharmacol., 175, 165-74 (1990)], human immunodeficiency virus (HIV) related neuronal injury [Annu. Rev. Pharmacol. Toxicol., 1998; 38159-77], amyotrophic lateral sclerosis [Neurol. Res., 21, 309-12 (1999)], tolerance and/or dependence to opioid treatment of pain [Brain. Res., 731, 171-181 (1996)], withdrawal syndromes of e.g., alcohol, opioids or cocaine [Drug and Alcohol Depend., 59, 1-15 (2000)], muscular spasm [Neurosci. Lett., 73, 143-148 (1987)], dementia of various origins [Expert Opin. Investig. Drugs, 9, 1397-406 (2000)]. An NMDA antagonist may also be useful to treat cerebral ischemia of any origin (e.g., stroke, heart surgery), chronic neurodegenerative disorders, such as Alzheimer's disease, Parkinson's disease, Huntington's disease, pain (e.g., posttraumatic or postoperative) and chronic pain states, such as neuropathic pain or cancer related pain, epilepsy, anxiety, depression, migraine, psychosis, hypoglycemia, degenerative disorders of the retina (e.g., CMV retinitis), glaucoma, asthma, tinnitus, aminoglycoside antibiotic-induced hearing loss [Drug News Perspect 11, 523-569 (1998) and International Publication No. WO 00/00197].

Accordingly, any of the above described solid forms of Radiprodil (anhydrate Form A, anhydrate Form B, dihydrate, monohydrate, and amorphous) may be beneficially used for the treatment of traumatic injury of brain or spinal cord, human immunodeficiency virus (HIV) related neuronal injury, amyotrophic lateral sclerosis, tolerance and/or dependence to opioid treatment of pain, withdrawal syndromes of e.g., alcohol, opioids or cocaine, ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss.

Any of the above described solid forms of 2-[4-(4-fluoro-benzyl)-piperidine-1-yl]-2-oxo-N-(2-oxo-2,3-dihydro-benzoxazole-6-yl)-acetamide (Radiprodil anhydrate Form A, anhydrate Form B, dihydrate, monohydrate, and amorphous) can normally be administered in a daily dosage regimen (for an adult patient) of, for example, between about 0.01 mg and about 200 mg, such as between 0.1 mg and about 100 mg, e.g. between about 1 mg and about 50 mg.

Any of the above described solid forms of 2-[4-(4-fluoro-benzyl)-piperidine-1-yl]-2-oxo-N-(2-oxo-2,3-dihydro-benzoxazole-6-yl)-acetamide (Radiprodil anhydrate Form A, anhydrate Form B, dihydrate, monohydrate, and amorphous) can be administered 1 to 4 times per day, for example, once a day, twice a day. The above described solid forms of Radprodil (anhydrate Form A, anhydrate Form B, dihydrate, monohydrate, and amorphous) can suitably be administered for a period of continuous therapy, for example for a week or more.

### Applied Measuring Conditions:

The X-ray powder diffraction measurements were performed on a PANalytical X′Pert PRO diffractometer using CuKa radiation in reflection geometry with 40 kV accelerating voltage and 40 mA anode current at a scanning rate of 0.02° 2Θ/s over the range of 2-40° 2θ, spinning the sample holder by 1 revolution/s.

FT-IR spectra were measured on a Thermo Nicolet 6700 FT-IR spectrometer in KBr pellets accumulating 100 scans at 4 cm⁻¹ spectral resolution in the range of 4000-400 cm⁻¹.
FT-Raman measurements were performed on a Perkin-Elmer Spectrum 2000 FT-Raman spectrometer equipped with a Nd:YAG laser operating at 1064 nm, and room temperature DTGS detector. The power of the irradiating beam was set to 300 mW; the applied scan number was 50 at 4 cm⁻¹ spectral resolution in the range of 3500-200 cm⁻¹.
DSC measurements were carried out on a TA Instruments DSC Q1000 equipped with RCS unit. Temperature and enthalpy calibration was done with indium and tin standards. Open aluminum pans were applied at 10 K/min heating rate with high purity nitrogen purge at 50 ml/min flow rate.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

### 2-[4-(4-Fluorobenzyl)-piperidin-1-yl]-2-oxo-N-(2-oxo-2,3-dihydro-benzoxazol-6-yl)-acetamide anhydrate Form A

To a stirred mixture of [4-(4-fluorobenzyl)-piperidin-1-yl]-oxoacetic acid (11.8 g, 0.045 mol), HBTU ((O-benzotriazol-1-yl-N,N,N′,N′-tetramethyl-uronium hexafluoro-phosphate), 16.85 g (0.045 mol)), triethylamine (4.55 g (6.24 ml, 0.045 mol)), and dimethylformamide (100 ml) 6-amino-3H-benzoxazol-2-one (5.6 g, 0.037 mol) was added. The solution was stirred for 2 h at room temperature, then 8% NaHCO₃ solution (136 ml) was added dropwise. The reaction mixture was stirred for 4 h, the precipitated crystals was filtered, washed with water (2×150 ml), dried on 50° C. to yield 15 g of the crude title compound.
The crude product was dissolved in a mixture of hot acetone (300 ml) and water (20 ml), evaporated to 150 ml and chilled to obtain 10.4 g of the title compound. Water content (Karl-Fischer): 0%. Mp.: 224-225° C.
XRPD reflections measured at: 7.8, 12.2, 13.8, 15.0, 16.7, 17.5, 18.7, 19.1, 22.0, 23.7, 27.0, 27.6 and 29.0±0.2° 2θ.
Absorption bands in IR spectra measured at: 3295, 2915, 2863, 1852, 1768, 1677, 1639, 1617, 1506, 1448, 1305, 1214, 1058, 964, 926, 816, 708, 635, 496±4 cm⁻¹.
Absorption bands in Raman spectra measured at: 3106, 3073, 2954, 2946, 2865, 1680, 1641, 1533, 1470, 1452, 1314, 1274, 1243, 1101, 968, 882, 850, 711, 639, 603, 507±4 cm⁻¹.

### Example 2

### 2-[4-(4-Fluorobenzyl)-piperidin-1-yl]-2-oxo-N-(2-oxo-2,3-dihydro-benzoxazol-6-yl)-acetamide anhydrate Form A

2-[4-(4-Fluorobenzyl)-piperidin-1-yl]-2-oxo-N-(2-oxo-2,3-dihydro-benzoxazol-6-yl)-acetamide obtained in Example 1 (1 g) was dissolved in dimethyl formamide (10 ml). To this solution 5% aqueous NaHCO₃ (10 ml) was added while stirring. After an additional hour the precipitated crystals were filtered, washed two times with water (7 ml), dried at 50° C. to yield 0.40 g of anhydrate. Water content (Karl-Fischer): 0.81%. Mp.: 223-225° C.
XRPD reflections measured at: 7.8, 12.2, 13.8, 15.0, 16.7, 17.5, 18.7, 19.1, 22.0, 23.7, 27.0, 27.6 and 29.0±0.2° 2θ.
Absorption bands in IR spectra measured at: 3295, 2915, 2863, 1852, 1768, 1677, 1639, 1617, 1506, 1448, 1305, 1214, 1058, 964, 926, 816, 708, 635, 496±4 cm⁻¹.
Absorption bands in Raman spectra measured at: 3106, 3073, 2954, 2946, 2865, 1680, 1641, 1533, 1470, 1452, 1314, 1274, 1243, 1101, 968, 882, 850, 711, 639, 603, 507±4 cm⁻¹.

### Example 3

### 2-[4-(4-Fluorobenzyl)-piperidin-1-yl]-2-oxo-N-(2-oxo-2,3-dihydro-benzoxazol-6-yl)-acetamide anhydrate Form A

2-[4-(4-Fluorobenzyl)-piperidin-1-yl]-2-oxo-N-(2-oxo-2,3-dihydro-benzoxazol-6-yl)-acetamide dihydrate (3 g) was dissolved in the mixture of acetone (60 ml) and water (4 ml) under reflux, then evaporated to 35 ml, cooled and stirred at 20° C. for 0.5 h and at 5° C. for 1 h, to yield 1.90 g of the title anhydrate. Water content (Karl-Fischer): 0%. Mp.: 223-224° C.
XRPD reflections measured at: 7.8, 12.2, 13.8, 15.0, 16.7, 17.5, 18.7, 19.1, 22.0, 23.7, 27.0, 27.6 and 29.0±0.2° 2θ.
Absorption bands in IR spectra measured at: 3295, 2915, 2863, 1852, 1768, 1677, 1639, 1617, 1506, 1448, 1305, 1214, 1058, 964, 926, 816, 708, 635, 496±4 cm⁻¹.
Absorption bands in Raman spectra measured at: 3106, 3073, 2954, 2946, 2865, 1680, 1641, 1533, 1470, 1452, 1314, 1274, 1243, 1101, 968, 882, 850, 711, 639, 603, 507±4 cm⁻¹.

### Example 4

### 2-[4-(4-Fluorobenzyl)-piperidin-1-yl]-2-oxo-N-(2-oxo-2,3-dihydro-benzoxazol-6-yl)-acetamide dihydrate

To a stirred mixture of [4-(4-fluorobenzyl)-piperidin-1-yl]-oxoacetic acid (11.8 g, 0.045 mol), HBTU ((O-benzotriazol-1-yl-N,N,N′,N′-tetramethyl-uronium hexafluoro-phosphate), 16.85 g (0.045 mol)), triethylamine (4.55 g (6.24 ml, 0.045 mol)) and dimethylformamide (100 ml), 6-amino-3H-benzoxazol-2-one (5.6 g, 0.037 mol) was added. The solution was stirred for 2 h at room temperature, then 8% NaHCO₃ solution (136 ml) was added dropwise. The mixture was stirred for 4 h, the precipitated crystals was filtered, washed twice with 150 ml of water. The wet product (45 g, dry content 33% of the title compound) was dissolved in acetone (300 ml) and added dropwise to a mixture of 1% NaHCO₃ solution (200 ml) and acetone (80 ml) below 10° C. The mixture was stirred for 1 h, washed three times with water (70 ml), dried at 50° C. to yield 8.5 g of dihydrate. Water content (Karl-Fischer): 8.3%. Mp.: 224-225° C.
XRPD reflections measured at: 3.6, 7.2, 10.8, 12.5, 14.6, 15.3, 16.1, 17.4, 19.0, 19.5, 21.3, 22.6, 24.0, 25.2, 26.1 and 28.3±0.2° 2θ.
Absorption bands in IR spectra measured at: 3426, 3335, 2950, 1870, 1776, 1668, 1633, 1593, 1509, 1447, 1221, 1197, 968, 939, 852, 815, 754±4 cm⁻¹.
Absorption bands in Raman spectra measured at: 3068, 2952, 2855, 1772, 1669, 1641, 1504, 1458, 1337, 1313, 1160, 1105, 964, 850, 707, 676, 638, 299±4 cm⁻¹.

### Example 5

### 2-[4-(4-Fluorobenzyl)-piperidin-1-yl]-2-oxo-N-(2-oxo-2,3-dihydro-benzoxazol-6-yl)-acetamide dihydrate

The solution of 2-[4-(4-Fluorobenzyl)-piperidin-1-yl]-2-oxo-N-(2-oxo-2,3-dihydro-benzoxazol-6-yl)-acetamide obtained in Example 1 (1 g) and dimethyl sulfoxide (2 ml) was added dropwise to 5% aqueous NaHCO₃ solution (30 ml). After stirring for 1 h the precipitated crystals were suspended in water (30 ml) and stirred for an additional hour, washed two times with water (7 ml), dried at 50° C. to yield 0.95 g of dihydrate. Water content (Karl-Fischer): 8.3%. Mp.: (106)-224-225° C. Purity: 99.5% (HPLC)
XRPD reflections measured at: 3.6, 7.2, 10.8, 12.5, 14.6, 15.3, 16.1, 17.4, 19.0, 19.5, 21.3, 22.6, 24.0, 25.2, 26.1 and 28.3±0.2° 2θ.
Absorption bands in IR spectra measured at: 3426, 3335, 2950, 1780, 1776, 1668, 1633, 1593, 1509, 1447, 1221, 1197, 968, 939, 852, 815, 754±4 cm⁻¹.
Absorption bands in Raman spectra measured at: 3068, 2952, 2855, 1772, 1669, 1641, 1504, 1458, 1337, 1313, 1160, 1105, 964, 850, 707, 676, 638, 299±4 cm⁻¹.

### Example 6

### 2-[4-(4-Fluorobenzyl)-piperidin-1-yl]-2-oxo-N-(2-oxo-2,3-dihydro-benzoxazol-6-yl)-acetamide dihydrate

The solution of 2-[4-(4-Fluorobenzyl)-piperidin-1-yl]-2-oxo-N-(2-oxo-2,3-dihydro-benzoxazol-6-yl)-acetamide obtained in Example 1 (1 g) and dimethyl formamide (10 ml) was added to 5% aqueous NaHCO₃ solution (10 ml) while stirring. After an additional hour the precipitated crystals were filtered, washed two times with water (7 ml), dried at 50° C. to yield 0.50 g of dihydrate. Water content (Karl-Fischer): 8.47%. Mp.: (106)-222-224° C.
XRPD reflections measured at: 3.6, 7.2, 10.8, 12.5, 14.6, 15.3, 16.1, 17.4, 19.0, 19.5, 21.3, 22.6, 24.0, 25.2, 26.1 and 28.3±0.2° 2θ.
Absorption bands in IR spectra measured at: 3426, 3335, 2950, 1870, 1776, 1668, 1633, 1593, 1509, 1447, 1221, 1197, 968, 939, 852, 815, 754±4 cm⁻¹.
Absorption bands in Raman spectra measured at: 3068, 2952, 2855, 1772, 1669, 1641, 1504, 1458, 1337, 1313, 1160, 1105, 964, 850, 707, 676, 638, 299±4 cm⁻¹.

### Example 7

### 2-[4-(4-Fluorobenzyl)-piperidin-1-yl]-2-oxo-N-(2-oxo-2,3-dihydro-benzoxazol-6-yl)-acetamide dihydrate

2-[4-(4-Fluorob enzyl)-piperidin-1-yl]-2-oxo-N-(2-oxo-2,3-dihydro-b enzoxazol-6-yl)-acetamide obtained in Example 1 (1 g) was dissolved in dioxane (20 ml) under reflux. The solution was added to 5% aqueous NaHCO₃ (15 ml) at 20-22° C. while stirring. After 1 h the separated crystals was filtered to yield 0.80 g of the title dihydrate. Water content (Karl-Fischer): 8.45%. Mp.: (108)-222-224° C.

XRPD reflections measured at: 3.6, 7.2, 10.8, 12.5, 14.6, 15.3, 16.1, 17.4, 19.0, 19.5, 21.3, 22.6, 24.0, 25.2, 26.1 and 28.3±0.2° 2θ.

Absorption bands in IR spectra measured at: 3426, 3335, 2950, 1870, 1776, 1668, 1633, 1593, 1509, 1447, 1221, 1197, 968, 939, 852, 815, 754±4 cm⁻¹.
Absorption bands in Raman spectra measured at: 3068, 2952, 2855, 1772, 1669, 1641, 1504, 1458, 1337, 1313, 1160, 1105, 964, 850, 707, 676, 638, 299±4 cm⁻¹.

### Example 8

### 2-[4-(4-Fluorobenzyl)-piperidin-1-yl]-2-oxo-N-(2-oxo-2,3-dihydro-benzoxazol-6-yl)-acetamide monohydrate

2-[4-(4-fluorobenzyl)-piperidin-1-yl]-2-oxo-N-(2-oxo-2,3-dihydro-benzoxazol-6-yl)-acetamide obtained in Example 1 (5 g) is dissolved in a solvent-mixture (150 ml acetone and 9 ml water) under reflux. The solution is filtered through celite and the solution is cooled to 35-40° C. The solution is concentrated under reduced pressure at not more than 40° C. up to 30-35 ml. The suspension is agitated at 20-25° C. for 30 min. and at 0-5° C. for 1 hour. The product is filtered and washed twice with 4 ml of cold acetone and dried at not more than 50° C. to yield 3.2 g of the title monohydrate. Water content (Karl-Fischer): 4.2%. Mp.: 224-225° C.
XRPD reflections measured at: 4.9, 9.8, 11.5, 15.1, 18.2, 20.0, 24.6, 25.3, 26.8 and 30.2±0.2° 2θ.
Absorption bands in IR spectra measured at: 3644, 3358, 3157, 1836, 1809, 1789, 1645, 1621, 1594, 1508, 1230, 1158, 919, 758, 710, 542±4 cm⁻¹.
Absorption bands in Raman spectra measured at: 3072, 2950, 2887, 1642, 1597, 1504, 1484, 1456, 1415, 1336, 1295, 1102, 883, 848, 726, 639±4 cm⁻¹.

### Example 9

### 2-[4-(4-Fluorobenzyl)-piperidin-1-yl]-2-oxo-N-(2-oxo-2,3-dihydro-benzoxazol-6-yl)-acetamide amorphous form

5 g 2-[4-(4-Fluorobenzyl)-piperidin-1-yl]-2-oxo-N-(2-oxo-2,3-dihydro-benzoxazol-6-yl)-acetamide dihydrate uniformly spread over a glass plate is placed in an oven at 100° C. for 20 min under nitrogen stream. No reflections on the XRPD pattern.
Absorption bands in IR spectra measured at: 3275, 2919, 2854, 1768, 1685, 1623, 1508, 1446, 1219, 1157, 965, 928, 809, 758, 708, 544, 496±4 cm⁻¹.
Absorption bands in Raman spectra measured at: 3071, 2936, 1687, 1640, 1530, 1472, 1452, 1414, 1322, 1280, 1101, 968, 852, 710, 639, 388±4 cm⁻¹.

### Example 10

### 2-[4-(4-Fluorobenzyl)-piperidin-1-yl]-2-oxo-N-(2-oxo-2,3-dihydro-benzoxazol-6-yl)-acetamide anhydrate Form B

0.5 g 2-[4-(4-Fluorobenzyl)-piperidin-1-yl]-2-oxo-N-(2-oxo-2,3-dihydro-benzoxazol-6-yl)-acetamide monohydrate Form spread over a glass plate is placed in an oven at 130° C. for 5 min under nitrogen stream.
XRPD reflections measured at: 5.4, 15.2, 16.5, 16.9, 19.3, 20.9, 26.2 and 26.7±0.2° 2θ.
Absorption bands in IR spectra measured at: 3297, 3138, 2924, 1752, 1680, 1633, 1633, 1618, 1508, 1594, 1449, 1215, 1193, 1158, 965, 926, 809, 709, 559, 497±4 cm⁻¹.
Absorption bands in Raman spectra measured at: 3072, 2927, 2877, 1684, 1642, 1545, 1476, 1452, 1315, 1284, 1246, 1102, 967, 887, 854, 711, 639±4 cm⁻¹.

### Example 11

### 2-[4-(4-Fluorobenzyl)-piperidin-1-yl]-2-oxo-N-(2-oxo-2,3-dihydro-benzoxazol-6-yl)-acetamide dihydrate

To a stirred mixture of [4-(4-fluorobenzyl)-piperidin-1-yl]-oxoacetic acid (7.5 kg, 28 mol), HBTU ((O-benzotriazol-1-yl-N,N,N′,N′-tetramethyl-uronium hexafluoro-phosphate), 10.3 kg (28 mol)), triethylamine 2.9 kg (3.9 l, 28 mol) and dimethylformamide (100 l), 6-amino-3H-benzoxazol-2-one (3.6 kg, 24 mol) was added. The solution was stirred for 2 h at room temperature, then 8% NaHCO₃ solution (86 l) was added gradually. The mixture was stirred for 4 h, the precipitated crystals was filtered, washed twice with 90 l of water. The wet product (18.5 kg, dry content 30% of the title compound) was dissolved in acetone (170 l) and added gradually to a mixture of 1% NaHCO₃ solution (120 l) and acetone (45 l) below 10° C. The mixture was stirred for 1 h, washed three times with water (45 l), dried at 50° C. to yield 5.1 kg of dihydrate. Water content (Karl-Fischer): 8.2%. Mp.: (106)-224-225° C. Purity: 99.48% (HPLC)
XRPD reflections measured at: 3.6, 7.2, 10.8, 12.5, 14.0, 14.6, 15.3, 16.1, 17.4, 18.1, 19.0, 19.5, 21.3, 22.6, 24.0, 25.2, 26.1 and 28.3±0.2° 2θ.
Absorption bands in IR spectra measured at: 3426, 3335, 2950, 1870, 1776, 1668, 1633, 1593, 1509, 1447, 1221, 1197, 968, 939, 852, 815, 754±4 cm⁻¹.
Absorption bands in Raman spectra measured at: 3068, 2952, 2855, 1772, 1669, 1641, 1504, 1458, 1337, 1313, 1160, 1105, 964, 850, 707, 676, 638, 299±4 cm⁻¹.

1. A crystalline or amorphous form selected from the group consisting of:
a crystalline anhydrate form A of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 7.8, 22.0, 23.7, 27.0 and 27.6±0.2° 2θ,
b) an X-ray powder diffraction pattern substantially in accordance with FIG. 1,
c) characteristic FT Raman absorption bands at about 3106, 1680, 1641, 1470, 1274 and 507±4 cm⁻¹, or
d) an FT Raman spectrum substantially in accordance with FIG. 3; a crystalline anhydrate form B of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 5.4, 12.9, 16.9 and 21.0±0.2° 2θ,
b) an X-ray powder diffraction pattern substantially in accordance with FIG. 6,
c) characteristic FT Raman absorption bands at about 2877, 1684, 1642, 1545, 1476 and 887±4 cm⁻¹, or
d) an FT Raman spectrum substantially in accordance with FIG. 8;
a crystalline hydrate form of 2-[4-(4-Fluorobenzyl)-piperidin-1-yl]-2-oxo-N-(2-oxo-2,3-dihydro-benzoxazol-6-yl)-acetamide *nH₂O, where n is equal to 0.5, 1, 1.5 or 2;
a crystalline dihydrate form of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 3.6, 14.7, 19.1, 25.2 and 28.3±0.2° 2θ,
b) an X-ray powder diffraction pattern substantially in accordance with FIG. 11,
c) characteristic FT Raman absorption bands at about 3068, 1772, 1669, 1641 and 1313±4 cm⁻¹, or
d) an FT Raman spectrum substantially in accordance with FIG. 13;
a crystalline monohydrate form of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 4.9, 15.1, 18.1, 26.8 and 30.2±0.2° 2θ,
b) an X-ray powder diffraction pattern substantially in accordance with FIG. 16,
c) characteristic FT Raman absorption bands at about 2887, 1642, 1484, 1295 and 726, ±4 cm⁻¹, or
d) an FT Raman spectrum substantially in accordance with FIG. 18; and
an amorphous form of 2-[4-(4-fluorobenzyl)-piperidin-1-yl]-2-oxo-N-(2-oxo-2,3-dihydro-benzoxazol-6-yl)-acetamide having
a) no X-ray powder diffraction reflections, only the typical ‘halo’ pattern,
b) characteristic FT Raman absorption bands at about 3071, 1687, 1640, 1414, 1280 and 852±4 cm⁻¹, or
c) an FT Raman spectrum substantially in accordance with FIG. 23.

2. 2-[4-(4-Fluorobenzyl)-piperidin-1-yl]-2-oxo-N-(2-oxo-2,3-dihydro-benzoxazol-6-yl)-acetamide in solid form, wherein the solid form contains at least 50% of the crystalline or amorphous form of claim 1, and the crystalline or amorphous form is not the crystalline hydrate form.

3. The crystalline or amorphous form of claim 1 which is crystalline anhydrate Form B of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 5.4, 12.9, 16.9 and 21.0±0.2° 2θ;
b) an X-ray powder diffraction pattern substantially in accordance with FIG. 6;
c) characteristic FT Raman absorption bands at about 2877, 1684, 1642, 1545, 1476 and 887±4 cm⁻¹; or
d) an FT Raman spectrum substantially in accordance with FIG. 8.

4. (canceled)

5. The crystalline or amorphous form of claim 1 which is crystalline hydrate form of 2-[4-(4-Fluorobenzyl)-piperidin-1-yl]-2-oxo-N-(2-oxo-2,3-dihydro-benzoxazol-6-yl)-acetamide *nH₂O, where n is equal to 0.5, 1, 1.5 or 2.

6. The crystalline or amorphous form of claim 1 which is crystalline dihydrate Form of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 3.6, 14.7, 19.1, 25.2 and 28.3±0.2° 2θ;
b) an X-ray powder diffraction pattern substantially in accordance with FIG. 11;
c) characteristic FT Raman absorption bands at about 3068, 1772, 1669, 1641 and 1313±4 cm⁻¹; or
d) an FT Raman spectrum substantially in accordance with FIG. 13.

7. (canceled)

8. The crystalline or amorphous form of claim 1 which is crystalline monohydrate Form of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 4.9, 15.1, 18.1, 26.8 and 30.2±0.2° 2θ;
b) an X-ray powder diffraction pattern substantially in accordance with FIG. 16;
c) characteristic FT Raman absorption bands at about 2887, 1642, 1484, 1295 and 726, ±4 cm⁻¹; or
d) an FT Raman spectrum substantially in accordance with FIG. 18.

9. (canceled)

10. The crystalline or amorphous form of claim 1 which is amorphous Form of 2-[4-(4-fluorobenzyl)-piperidin-1-yl]-2-oxo-N-(2-oxo-2,3-dihydro-benzoxazol-6-yl)-acetamide having
a) no X-ray powder diffraction reflections, only the typical ‘halo’ pattern;
c) characteristic FT Raman absorption bands at about 3071, 1687, 1640, 1414, 1280 and 852±4 cm⁻¹; or
c) an FT Raman spectrum substantially in accordance with FIG. 23.

11. (canceled)

12. A process for the preparation of the crystalline or amorphous form of claim 1, said process comprising
evaporating or cooling a solution of 2-[4-(4-fluorobenzyl)-piperidin-1-yl]-2-oxo-N-(2-oxo-2,3-dihydro-benzoxazol-6-yl)-acetamide in a mixture of water and water miscible organic solvent is evaporated or cooled,
adding dilute NaHCO₃ solution in the solution of 2-[4-(4-fluorobenzyl)-piperidin-1-yl]-2-oxo-N-(2-oxo-2,3-dihydro-benzoxazol-6-yl)-acetamide in a water miscible organic solvent,
heating up to 130° C. and then cooling to room temperature temperature the crystalline monohydrate form of 2-[4-(4-fluorobenzyl)-piperidin-1-yl]-2-oxo-N-(2-oxo-2,3-dihydro-benzoxazol-6-yl)-acetamide,
adding the solution of 2-[4-(4-fluorobenzyl)-piperidin-1-yl]-2-oxo-N-(2-oxo-2,3-dihydro-benzoxazol-6-yl)-acetamide in a water miscible organic solvent to dilute NaHCO₃ solution
first cooling to a temperature of from 45 to 35° C., then to ambient temperature, finally to a temperature of from 5 to 0° C., the solution of the 2-[4-(4-fluorobenzyl)-piperidin-1-yl]-2-oxo-N-(2-oxo-2,3-dihydro-benzoxazol-6-yl)-acetamide in acetone containing 5-10% of water at reflux, or
drying the dihydrate form of 2-[4-(4-fluorobenzyl)-piperidin-1-yl]-2-oxo-N-(2-oxo-2,3-dihydro-benzoxazol-6-yl)-acetamide at about 100° C. under vacuum or dry nitrogen stream.

13. The process of claim 12 wherein the water miscible organic solvent is acetone

14. The process of claim 12 wherein the crystalline anhydrate Form A of the 2-[4-(4-fluorobenzyl)-piperidin-1-yl]-2-oxo-N-(2-oxo-2,3-dihydro-benzoxazol-6-yl)-acetamide is prepared by adding dilute NaHCO₃ solution in the solution of 2-[4-(4-fluorobenzyl)-piperidin-1-yl]-2-oxo-N-(2-oxo-2,3-dihydro-benzoxazol-6-yl)-acetamide in a water miscible organic solvent.

15. (canceled)

16. The process of claim 12 wherein the crystalline anhydrate Form B of the 2-[4-(4-fluorobenzyl)-piperidin-1-yl]-2-oxo-N-(2-oxo-2,3-dihydro-benzoxazol-6-yl)-acetamide is prepared by heating up to 130° C. and then cooling to room temperature the crystalline monohydrate form of 2-[4-(4-fluorobenzyl)-piperidin-1-yl]-2-oxo-N-(2-oxo-2,3-dihydro-benzoxazol-6-yl)-acetamide.

17. The process of claim 12 wherein the crystalline dihydrate Form of the 2-[4-(4-fluorobenzyl)-piperidin-1-yl]-2-oxo-N-(2-oxo-2,3-dihydro-benzoxazol-6-yl)-acetamide is prepared by adding the solution of 2-[4-(4-Fluorobenzyl)-piperidin-1-yl]-2-oxo-N-(2-oxo-2,3-dihydro-benzoxazol-6-yl)-acetamide in a water miscible organic solvent is added to dilute NaHCO₃ solution.

18. (canceled)

19. The process according to claim 17, wherein the dilute NaHCO₃ solution contains 25-30% of acetone.

20. The process of claim 12 wherein the crystalline monohydrate Form of the 2-[4-(4-fluorobenzyl)-piperidin-1-yl]-2-oxo-N-(2-oxo-2,3-dihydro-benzoxazol-6-yl)-acetamide is prepared by first cooling to a temperature of from 45 to 35° C., then to ambient temperarure, finally to a temperature of from 5 to 0° C., the solution of the 2-[4-(4-fluorobenzyl)-piperidin-1-yl]-2-oxo-N-(2-oxo-2,3-dihydro-benzoxazol-6-yl)-acetamide in acetone containing 5-10% of water at reflux.

21. The process of claim 12 wherein the amorphous Form of the 2-[4-(4-fluorobenzyl)-piperidin-1-yl]-2-oxo-N-(2-oxo-2,3-dihydro-benzoxazol-6-yl)-acetamide is prepared by drying the dihydrate form of 2-[4-(4-fluorobenzyl)-piperidin-1-yl]-2-oxo-N-(2-oxo-2,3-dihydro-benzoxazol-6-yl)-acetamide at about 100° C. under vacuum or dry nitrogen stream.

22. (canceled)

23. (canceled)

24. A method for treating or preventing a condition which requires modulation of an NMDA receptor comprising administering to a patient in need thereof, an effective amount of the crystalline or amorphous form of claim 1.

25. The method according to claim 24, wherein the NMDA receptor is an NR2B selective NMNDA receptor.

26. (canceled)

27. (canceled)

28. The crystalline or amorphous form of claim 1 which is crystalline anhydrate form A of 2-[4-(4-fluorobenzyl)-piperidin-1-yl]-2-oxo-N-(2-oxo-2,3-dihydro-benzoxazol-6-yl)-acetamide having
a) characteristic X-ray powder diffraction reflections at about 7.8, 22.0, 23.7, 27.0 and 27.6±0.2° 2θ,
b) an X-ray powder diffraction pattern substantially in accordance with FIG. 1,
c) characteristic FT Raman absorption bands at about 3106, 1680, 1641, 1470, 1274 and 507±4 cm⁻¹, or
d) an FT Raman spectrum substantially in accordance with FIG. 3.
